(19) United States
(12) Patent Application Publication
(10) Pub. No.: US 2024/0324961 A1
(43) Pub. Date: Oct. 3, 2024

(54) BIOLOGICAL INFORMATION MEASUREMENT APPARATUS AND BIOLOGICAL INFORMATION MEASUREMENT SET

(71) Applicant: SEIKO EPSON CORPORATION, TOKYO (JP)

(72) Inventors: Hiroki KOBAYASHI, HARA-MURA (JP); Ayae SAWADO, Kai-shi (JP); Yuichi TAKANO, SHIOJIRI-SHI NAGANO-KEN JAPAN (JP); Fumio KOYAMA, Hara-Mura (JP)

(21) Appl. No.: 18/620,008

(22) Filed: Mar. 28, 2024

(30) Foreign Application Priority Data
Mar. 29, 2023 (JP) 2023-052979

(51) Int. Cl. A61B 5/00; A61B 5/021; A61B 5/11; A61B 5/1455

(52) U.S. Cl. CPC A61B 5/721; A61B 5/02108; A61B 5/1116; A61B 5/1118; A61B 5/14552; A61B 5/6802; A61B 5/6824; A61B 5/6832; A61B 2560/0462; A61B 2562/0219; A61B 2562/0238; A61B 2562/046

(57) ABSTRACT

A biological information measurement apparatus includes: a detection unit including a light-emitting unit and a light-receiving unit; an inertial sensor configured to detect a body motion of a test subject; and a housing having a measurement surface configured to face the test subject and accommodating the detection unit and the inertial sensor, in which in a plan view of the measurement surface, a first width of the housing along a parallel axis parallel to an axis of the inertial sensor is different from a second width of the housing along an orthogonal axis orthogonal to the axis of the inertial sensor.

The present application is based on, and claims priority from JP Application Serial Number 2023-052979, filed Mar. 29, 2023, the disclosure of which is hereby incorporated by reference herein in its entirety.

### BACKGROUND

### 1. Technical Field

The present disclosure relates to a biological information measurement apparatus and a biological information measurement set.

### 2. Related Art

A biological information measurement apparatus that measures biological information such as a pulse wave and an oxygen saturation concentration is known. JP-A-2023-20895 discloses a pulse wave sensor as an example of a biological information measurement apparatus. The pulse wave sensor is attached to an upper arm portion, vicinity of a clavicle, a foot, or the like of a test subject. The pulse wave sensor includes a built-in acceleration sensor that is an example of an inertial sensor. The pulse wave sensor transmits a measurement result of the acceleration sensor to a control device. The control device calculates a relative positional correlation between the pulse wave sensor and a heart, and the likes, based on the measurement result of the acceleration sensor.

JP-A-2023-20895 is an example of the related art.

When the test subject wears the biological information measurement apparatus including the acceleration sensor, it is difficult for the test subject to wear the biological information measurement apparatus with a direction of an axis of the acceleration sensor in a desired direction.

### SUMMARY

A biological information measurement apparatus according to the present disclosure includes: a detection unit including a light-emitting unit and a light-receiving unit; an inertial sensor configured to detect a body motion of a test subject; and a housing having a measurement surface configured to face the test subject and accommodating the detection unit and the inertial sensor, in which in a plan view of the measurement surface, a first width of the housing along a parallel axis parallel to an axis of the inertial sensor is different from a second width of the housing along an orthogonal axis orthogonal to the axis of the inertial sensor.

A biological information measurement set according to the present disclosure includes: a biological information measurement apparatus including a detection unit including a light-emitting unit and a light-receiving unit, an inertial sensor configured to detect a body motion of a test subject, and a housing having a measurement surface configured to face the test subject and accommodating the detection unit and the inertial sensor; and a wearing tool configured to cover the housing and press the biological information measurement apparatus to the test subject, in which in a plan view of the measurement surface, a first width of the housing along a parallel axis parallel to an axis of the inertial sensor is different from a second width of the housing along an orthogonal axis orthogonal to the axis of the inertial sensor.

### BRIEF DESCRIPTION OF THE DRAWINGS

FIG. 1 is a diagram illustrating a schematic configuration of a measurement apparatus.

FIG. 2 is a diagram illustrating a schematic configuration of a detection surface of the measurement apparatus.

FIG. 3 is a diagram illustrating a block configuration of the measurement apparatus.

FIG. 4 is a diagram illustrating a schematic configuration of a motion sensor.

FIG. 5 is a plan view of a detection surface of a measurement apparatus.

FIG. 6 is a plan view of a detection surface of a measurement apparatus.

FIG. 7 is a plan view of a detection surface of a measurement apparatus.

FIG. 8 is a plan view of a detection surface of a measurement apparatus.

FIG. 9 is a plan view of a side surface of a measurement apparatus.

FIG. 10 is a plan view of a side surface of a measurement apparatus.

FIG. 11 is a diagram illustrating the measurement apparatus worn by a user.

FIG. 12 is a diagram illustrating the measurement apparatus worn by the user.

### DESCRIPTION OF EMBODIMENTS

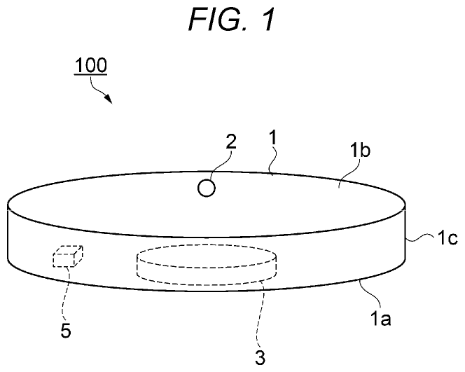

FIG. 1 illustrates a schematic configuration of a measurement apparatus 100. The measurement apparatus 100 measures biological information or various types of data related to the biological information on a user M such as a human. The user M corresponds to an example of a test subject. The measurement apparatus 100 is a coin-type portable apparatus worn by the user M at a measurement site. The measurement apparatus 100 illustrated in FIG. 1 is attached to, for example, an upper arm portion of the user M. The measurement apparatus 100 measures the biological information such as a pulse wave including a pulse interval and the like, an oxygen saturation concentration, and the like over time. The pulse wave interval is expressed as a post pacing interval (PPI). The pulse wave indicates a temporal change in volume of a blood vessel in conjunction with heartbeats. The oxygen saturation concentration indicates a proportion of hemoglobin bound to oxygen among hemoglobin in arterial blood of the user M. The oxygen saturation concentration is an index for evaluating a respiratory function of the user M. The measurement apparatus 100 may measure biological information other than the pulse wave and the oxygen saturation concentration. For example, the measurement apparatus 100 measures an arterial blood glucose concentration, an arterial blood alcohol concentration, or the like. The measurement apparatus 100 corresponds to an example of a biological information measurement apparatus. The measurement apparatus 100 includes a case 1 and a power button 2. The case 1 accommodates an optical detection unit 3 and a motion sensor 5. The case 1 is an exterior accommodating units and the like provided in the measurement apparatus 100. The case 1 accommodates the optical detection unit 3 and the motion sensor 5. The case 1 accommodates a control unit 30, a memory 40, a battery 70, and the like. The control unit 30, the memory 40, and the battery 70 will be described later. The case 1 has a detection surface 1a, an exterior surface 1b, and a side surface 1c. The case 1 corresponds to an example of a housing.

The detection surface 1a is a surface facing the measurement site of the user M. At least a part of the detection surface 1a comes into contact with the measurement site of the user M. The detection surface 1a is provided with the optical detection unit 3. A functional layer such as an adhesive layer 1d to be described later may be provided at the detection surface 1a. The detection surface 1a corresponds to an example of a measurement surface.

The exterior surface 1b is a surface facing the detection surface 1a. The exterior surface 1b comes into contact with a sleeve 200 to be described later when the user M wears the sleeve 200 at the measurement site. The power button 2 is provided at the exterior surface 1b. The exterior surface 1b corresponds to an example of a facing surface.

The side surface 1c couples the detection surface 1a and the exterior surface 1b. The side surface 1c is gripped when the user M wears the measurement apparatus 100 at the measurement site. The side surface 1c may be formed integrally with the exterior surface 1b. The side surface 1c may be formed integrally with a part of the detection surface 1a.

The power button 2 is operated by the user M. When the user M operates the power button 2, the measurement apparatus 100 starts or ends the measurement of the biological information. The power button 2 illustrated in FIG. 1 is provided at the exterior surface 1b, and is not limited thereto. The power button 2 may be provided at the detection surface 1a or the side surface 1c.

The optical detection unit 3 is disposed at the detection surface 1a of the case 1. The optical detection unit 3 is disposed at a position facing the measurement site of the user M. The optical detection unit 3 acquires various types of data used when measuring the biological information. The optical detection unit 3 corresponds to an example of a detection unit.

The motion sensor 5 detects a body motion of the user M. The motion sensor 5 is accommodated inside the case 1. The motion sensor 5 detects data relating to a motion, a posture, and the like of the user M.

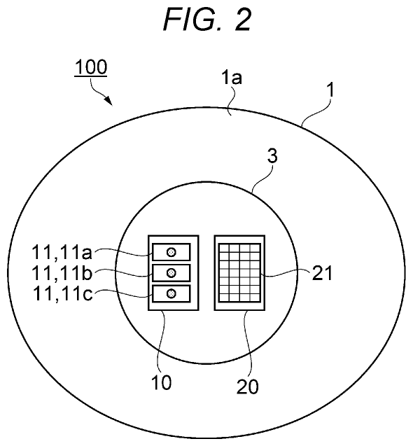

FIG. 2 illustrates a schematic configuration of the detection surface 1a of the measurement apparatus 100. FIG. 2 is a plan view of the detection surface 1a of the measurement apparatus 100. The optical detection unit 3 is disposed at the detection surface 1a. The optical detection unit 3 includes a light-emitting element unit 10 and a light-receiving element unit 20. The light-emitting element unit 10 corresponds to an example of a light-emitting unit. The light-receiving element unit 20 corresponds to an example of a light-receiving unit.

The light-emitting element unit 10 emits light toward the measurement site of the user M. The light-emitting element unit 10 illustrated in FIG. 2 includes three light-emitting elements 11. The three light-emitting elements 11 include a red-light-emitting element 11a, an infrared-light-emitting element 11b, and a green-light-emitting element 11c. The red-light-emitting element 11a, the infrared-light-emitting element 11b, and the green-light-emitting element 11c emit lights having different wavelength ranges. An arrangement of the three light-emitting elements 11 is appropriately set. The number of the light-emitting elements 11 is not limited to three. Two or less or four or more light-emitting elements 11 may be provided in the light-emitting element unit 10.

The light-emitting element 11 is implemented by a bare chip type or shell type LED (light emitting diode). The light-emitting element 11 may be implemented by a laser diode. A configuration of the light-emitting element 11 is appropriately set according to a wavelength range of an emitted light.

The red-light-emitting element 11a emits red light RL toward the measurement site of the user M. The red-light-emitting element 11a emits the red light RL in a wavelength range of 600 nm to 800 nm toward the measurement site. The red light RL is, for example, light having a peak wavelength of 660 nm.

The infrared-light-emitting element 11b emits infrared light NL toward the measurement site of the user M. The infrared-light-emitting element 11b emits the infrared light NL in a wavelength range of 800 nm to 1300 nm toward the measurement site. The infrared light NL is, for example, near-infrared light having a peak wavelength of 905 nm.

The green-light-emitting element 11c emits green light GL toward the measurement site of the user M. The green-light-emitting element 11c emits the green light GL in a wavelength range of 520 nm to 550 nm toward the measurement site. The green light GL is, for example, light having a peak wavelength of 520 nm.

The light-receiving element unit 20 receives the various lights emitted from the light-emitting element unit 10. The light-receiving element unit 20 includes a light-receiving element 21 that receives the various lights. The light-receiving element 21 receives transmitted light or reflected light of the lights emitted from the light-emitting element unit 10. The transmitted light is light transmitted through the user M. The reflected light is light reflected inside the user M and transmitted through an inside of the user M. The light-receiving element 21 includes one or more photodiodes.

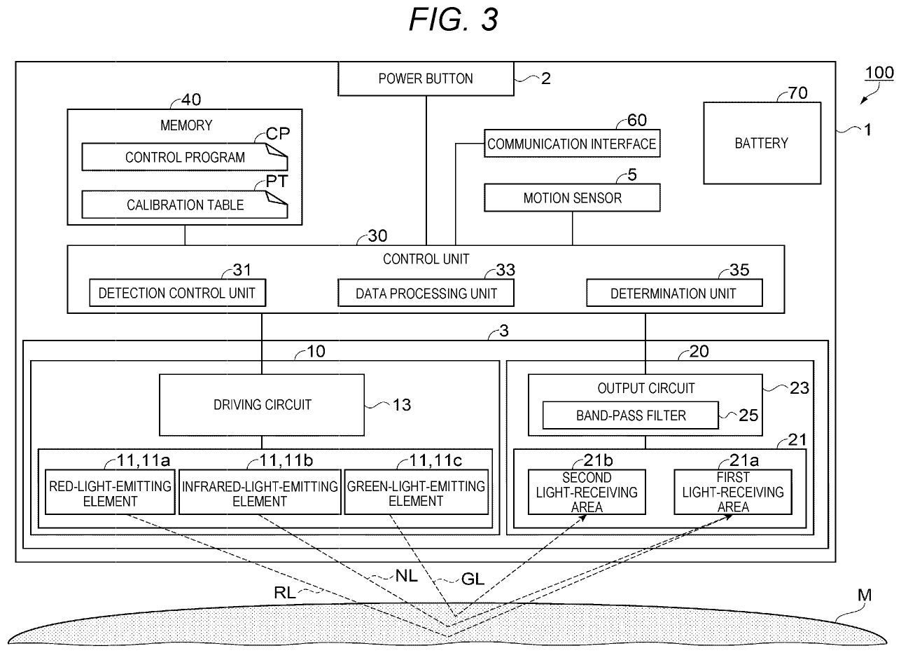

FIG. 3 illustrates a block configuration of the measurement apparatus 100. The measurement apparatus 100 accommodates various units and the like in the case 1. The measurement apparatus 100 includes the power button 2, the optical detection unit 3, the motion sensor 5, the control unit 30, the memory 40, a communication interface 60, and the battery 70.

The power button 2 supplies or interrupts power of the battery 70 to the optical detection unit 3, the control unit 30, and the like. The power button 2 may be a mechanical mechanism or a software switch. The user M can identify the detection surface 1a and the exterior surface 1b by touching the power button 2.

The optical detection unit 3 is an optical sensor module that detects data related to the biological information measured by using lights having various wavelength ranges as a detection signal. The optical detection unit 3 includes the light-emitting element unit 10 and the light-receiving element unit 20.

The light-emitting element unit 10 includes the red-light-emitting element 11a, the infrared-light-emitting element 11b, the green-light-emitting element 11c, and a driving circuit 13. The red-light-emitting element 11a, the infrared-light-emitting element 11b, and the green-light-emitting element 11c are disposed at positions facing the measurement site of the user M. The light-emitting element unit 10 operates under control of the control unit 30.

The driving circuit 13 drives a plurality of the light-emitting elements 11. The driving circuit 13 causes the plurality of light-emitting elements 11 to emit light under the control of the control unit 30. The driving circuit 13 illustrated in FIG. 3 causes the red-light-emitting element 11a, the infrared-light-emitting element 11b, and the green-light-emitting element 11c to emit light.

The light-receiving element unit 20 includes the light-receiving element 21 and an output circuit 23. The light-receiving element unit 20 operates under the control of the control unit 30. The light-receiving element 21 receives the light emitted by the light-emitting element 11 and reflected by the measurement site of the user M. The light-receiving element 21 generates a light-receiving signal corresponding to an intensity of the received light. The light-receiving element 21 transmits the light-receiving signal to the output circuit 23. The light-receiving element 21 receives the red light RL, the infrared light NL, and the green light GL reflected by the measurement site of the user M. The light-receiving element 21 is divided into a plurality of regions. The light-receiving element 21 may be divided into a plurality of regions by using an optical filter (not illustrated). The light-receiving element 21 illustrated in FIG. 3 is divided into a first light-receiving area 21a and a second light-receiving area 21b.

The first light-receiving area 21a receives the red light RL and the infrared light NL. The first light-receiving area 21a receives the red light RL emitted by the red-light-emitting element 11a and reflected by the measurement site of the user M. The first light-receiving area 21a receives the infrared light NL emitted by the infrared-light-emitting element 11b and reflected by the measurement site of the user M. The first light-receiving area 21a may receive at least one of the red light RL or the infrared light NL via the optical filter. The first light-receiving area 21a may alternately receive the red light RL and the infrared light NL in a time-division manner.

The second light-receiving area 21b receives the green light GL. The second light-receiving area 21b receives the green light GL emitted by the green-light-emitting element 11c and reflected by the measurement site of the user M. The second light-receiving area 21b may receive the green light GL via the optical filter.

The light-receiving element 21 illustrated in FIG. 3 receives the red light RL and the infrared light NL in the first light-receiving area 21a, and is not limited thereto. A third light-receiving area different from the first light-receiving area 21a and the second light-receiving area 21b may be provided. The red light RL or the infrared light NL may be received in the third light-receiving area. At this time, when the infrared light NL is received in the third light-receiving area, the first light-receiving area 21a receives the red light RL. The light-receiving element 21 may not be divided into the plurality of regions. The light-receiving element 21 may receive the red light RL, the infrared light NL, and the green light GL in a time-division manner.

The output circuit 23 generates a detection signal based on the light-receiving signal received from the light-receiving element 21 and outputs the detection signal to the control unit 30. The output circuit 23 generates the detection signal by performing processing such as analog-digital conversion on the light-receiving signal. The output circuit 23 generates a red light detection signal based on a red-light-receiving signal generated in the first light-receiving area 21a. The output circuit 23 generates an infrared light detection signal based on an infrared-light-receiving signal generated in the first light-receiving area 21a. The output circuit 23 generates a green light detection signal based on a green-light-receiving signal generated in the second light-receiving area 21b.

The output circuit 23 includes a band-pass filter 25. The band-pass filter 25 extracts an AC component from the light-receiving signal. The band-pass filter 25 separates the AC component and a DC component by extracting the AC component from the light-receiving signal. The band-pass filter 25 outputs the separated AC component and DC component to the control unit 30 as the detection signal. The measurement apparatus 100 illustrated in FIG. 3 includes the band-pass filter 25, and is not limited thereto. The output circuit 23 may not include the band-pass filter 25.

The control unit 30 is a controller that controls operations of various units. The control unit 30 is, for example, a processor including a central processing unit (CPU). The control unit 30 is implemented by one or more processors. The control unit 30 may include a semiconductor memory such as a random access memory (RAM) or a read only memory (ROM). The semiconductor memory functions as a work area of the control unit 30. The control unit 30 functions as a detection control unit 31, a data processing unit 33, and a determination unit 35 by executing a control program CP stored in the memory 40. The detection control unit 31, the data processing unit 33, and the determination unit 35 are functional units. The control unit 30 causes the light-emitting element unit 10 and the light-receiving element unit 20 to operate under control of the functional units.

The detection control unit 31 controls the light-emitting element unit 10 and the light-receiving element unit 20. The detection control unit 31 causes the light-emitting element unit 10 and the light-receiving element unit 20 to operate. The detection control unit 31 controls a light emission timing and a turn-off timing of the light-emitting element 11, adjusts a light amount thereof via the driving circuit 13, and the likes. The detection control unit 31 controls a light-receiving timing, a light-receiving time, a digital-analog conversion, and the like of various lights on the light-receiving element unit 20. The detection control unit 31 controls the light-emitting element unit 10 and the light-receiving element unit 20 to cause the optical detection unit 3 to generate the red light detection signal, the infrared light detection signal, and the green light detection signal. The detection control unit 31 controls the light-emitting element unit 10 and the light-receiving element unit 20 based on a determination result of the determination unit 35.

The detection control unit 31 may acquire detection data detected by the motion sensor 5. The detection control unit 31 controls operations of the light-emitting element unit 10 and the light-receiving element unit 20 based on the detection data. The detection control unit 31 controls the light emission timing or the turn-off timing of each light-emitting element 11 based on the detection data. The detection control unit 31 controls an output timing of each detection signal based on the detection data.

The data processing unit 33 processes each detection signal output from the light-receiving element unit 20. The data processing unit 33 acquires the red light detection signal, the infrared light detection signal, and the green light detection signal from the light-receiving element unit 20.

The data processing unit 33 performs a time-frequency analysis such as a short-time Fourier transform on the detection signal. The data processing unit 33 performs a frequency analysis on the detection signal by performing the time-frequency analysis. The data processing unit 33 calculates the biological information such as the pulse wave and the oxygen saturation concentration, or the data related to the biological information based on the detection signal. For example, the data processing unit 33 calculates the oxygen saturation concentration by using the red light detection signal, the infrared light detection signal, and calibration data PT. The data processing unit 33 calculates the pulse wave by using the green light detection signal. The data processing unit 33 may analyze arrhythmia, sleep apnea syndrome, and the like by using the calculated biological information. The data processing unit 33 transmits calculation data such as the biological information, analysis data, and the like to an external device via the communication interface 60. The data processing unit 33 may store the calculation data such as the biological information, the analysis data, and the like in the memory 40.

The data processing unit 33 may acquire the detection data detected by the motion sensor 5. The data processing unit 33 determines reliability of the calculation data, corrects the analysis data, and the likes, based on the detection data.

The determination unit 35 acquires the detection data detected by the motion sensor 5. The determination unit 35 determines the body motion, a state, the posture, and the like of the user M based on the detection data. The determination unit 35 determines an intensity of the body motion of the user M by using the detection data. The determination unit 35 determines whether the user M is sleeping or active by using the detection data. The determination unit 35 determines whether the user M is standing or lying, and the likes, by using the detection data. The determination unit 35 may output an identification signal corresponding to the state of the user M to the communication interface 60 and the like.

The memory 40 stores various types of data. The memory 40 stores control data for causing various units to operate, various types of data calculated by the control unit 30, and the like. The memory 40 may store various types of evaluation data used by the determination unit 35 and the like. The memory 40 may store the pulse wave, the oxygen saturation concentration, and the like calculated by the data processing unit 33. The memory 40 stores the control program CP that operates in the control unit 30. The memory 40 stores a calibration table PT used in the data processing unit 33. The memory 40 may store a conversion formula or a conversion table. The memory 40 is implemented by an ROM, an RAM, or the like.

The control program CP causes various functional units to operate by being executed by the control unit 30. The control program CP causes the control unit 30 to operate as the detection control unit 31, the data processing unit 33, and the determination unit 35. The control program CP may cause the control unit 30 to operate as functional units other than the detection control unit 31, the data processing unit 33, and the determination unit 35.

The calibration table PT is a table storing a fluctuation component amplitude ratio and the oxygen saturation concentration in association with each other. The fluctuation component amplitude ratio is a ratio between a red light transmission amount and an infrared light transmission amount. The red light transmission amount is a light amount of the red light RL emitted from the red-light-emitting element 11a, passing through the measurement site of the user M, and reaching the light-receiving element 21. The infrared light transmission amount is a light amount of the infrared light NL emitted from the infrared-light-emitting element 11b, passing through the measurement site of the user M, and reaching the light-receiving element 21. The red light transmission amount is calculated based on the red light detection signal. The infrared light transmission amount is calculated based on the infrared light a detection signal. The calibration table PT indicates correlation between the fluctuation component amplitude ratio and the oxygen saturation concentration. The calibration table PT is created in advance by a manufacturer of the measurement apparatus 100. The data processing unit 33 determines the oxygen saturation concentration corresponding to the calculated fluctuation component amplitude ratio by referring to the calibration table PT.

The memory 40 may store a calibration formula instead of the calibration table PT. The calibration formula is a relational formula between the fluctuation component amplitude ratio and the oxygen saturation concentration. The data processing unit 33 calculates the oxygen saturation concentration corresponding to the calculated fluctuation component amplitude ratio by using the calibration formula.

The motion sensor 5 detects a body motion of the user M. The motion sensor 5 generates detection data indicating the body motion of the user M. The motion sensor 5 outputs the generated detection data to the control unit 30. Examples of the motion sensor 5 include an acceleration sensor, an orientation sensor, and a gyro sensor. The motion sensor 5 may be an acceleration sensor. The acceleration sensor detects a movement, a posture, and the like of the measurement site to which the measurement apparatus 100 is attached. The motion sensor corresponds to an example of an inertial sensor.

The communication interface 60 is an interface circuit communicably connected with the external device. The communication interface 60 is coupled to the external device in a wired or wireless manner according to a predetermined protocol. The communication interface 60 includes, for example, a coupling port for wired communication, an antenna for wireless communication, or the like. The communication interface 60 receives the control data, information related to the user M, and the like from the external device. The communication interface 60 transmits the biological information such as the pulse wave and the oxygen saturation concentration, the data related to the biological information, and the like to the external device. The communication interface 60 transmits various types of measurement data such as the red light detection signal and the infrared light detection signal. The communication interface 60 transmits the detection data detected by the motion sensor 5 to the external device.

The battery 70 supplies the power to various units and the like of the measurement apparatus 100. The battery 70 includes a lithium primary battery, a lithium-ion secondary battery, or the like. The battery 70 may be a rechargeable lithium-ion secondary battery. The lithium-ion secondary battery is charged in a wired or wireless manner.

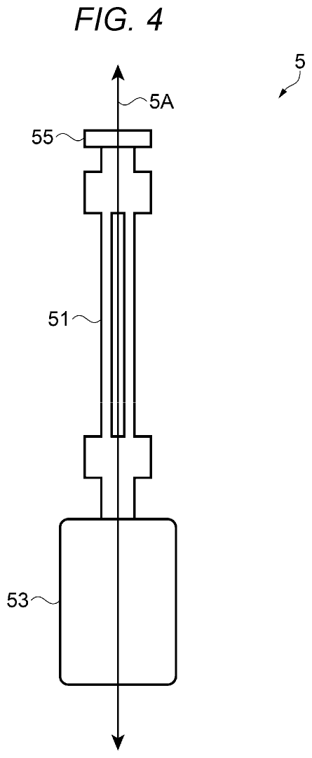

FIG. 4 illustrates a schematic configuration of the motion sensor 5. FIG. 4 illustrates an example of the motion sensor 5. A configuration of the motion sensor 5 is not limited to the configuration illustrated in FIG. 4. The motion sensor 5 includes a crystal vibrator 51, a weight 53, and a fixing member 55. FIG. 4 illustrates an axis 5A of the motion sensor 5.

The crystal vibrator 51 functions as a spring. When an acceleration is applied along the axis 5A, a stress is applied to the crystal vibrator 51. When the stress is applied to the crystal vibrator 51, a resonance frequency of the crystal vibrator 51 changes. When a sensor (not illustrated) detects a change in resonance frequency, the acceleration along the axis 5A is detected. The crystal vibrator 51 illustrated in FIG. 4 is a crystal double-tuning fork vibrator. The crystal double-tuning fork vibrator is implemented by coupling two tuning fork vibrators.

The weight 53 is coupled to one end of the crystal vibrator 51. The weight 53 is disposed at a free end of the crystal vibrator 51. When the acceleration is applied along the axis 5A, a position of the weight 53 fluctuates. When the position of the weight 53 fluctuates, the crystal vibrator 51 vibrates along the axis 5A.

The fixing member 55 fixes the other end of the crystal vibrator 51. The fixing member 55 fixes the other end, which is an end portion opposite to the one end of the crystal vibrator 51 that supports the weight 53 along the axis 5A. The other end of the crystal vibrator 51 fixed by the fixing member 55 serves as a fixed end. The fixing member 55 may be a part of an accommodating container that accommodates the crystal vibrator 51 and the weight 53, or may be a support member supported by the accommodating container.

The axis 5A is a virtual axis corresponding to a direction of an acceleration measured by the motion sensor 5. When a direction of the axis 5A is set to a desired direction, measurement accuracy of the posture, the body motion, and the like of the user M is improved.

The motion sensor 5 may be a one-axis acceleration sensor or a three-axis acceleration sensor. The motion sensor 5 may be a six-axis acceleration sensor. When the three-axis acceleration sensor or the six-axis acceleration sensor is used, the axis 5A indicates one of a plurality of axes.

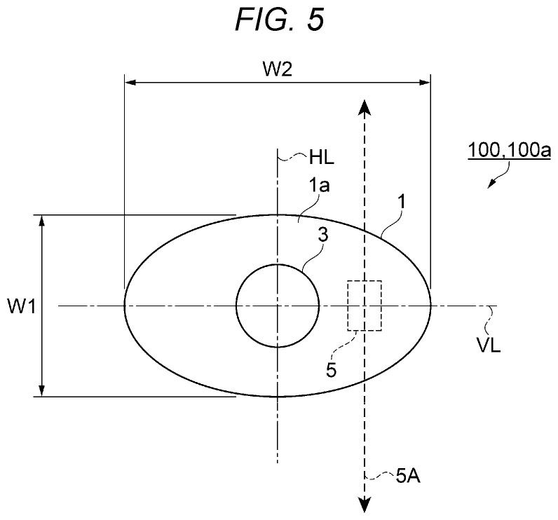

FIG. 5 is a plan view of the detection surface 1a of the measurement apparatus 100. FIG. 5 illustrates a first measurement apparatus 100a that is an example of the measurement apparatus 100. FIG. 5 illustrates the detection surface 1a of the case 1 and the optical detection unit 3. In the first measurement apparatus 100a, the motion sensor 5 is accommodated in the case 1. FIG. 5 illustrates the axis 5A, a virtual horizontal axis HL, and a virtual vertical axis VL. In FIG. 5, the light-emitting element unit 10 and the light-receiving element unit 20 in the optical detection unit 3 are omitted.

The virtual horizontal axis HL is a virtual axis parallel to the axis 5A of the motion sensor 5. The virtual horizontal axis HL corresponds to an example of a parallel axis. The virtual vertical axis VL is a virtual axis orthogonal to the axis 5A of the motion sensor 5. The virtual vertical axis VL corresponds to an example of an orthogonal axis.

In the plan view of the detection surface 1a, the detection surface 1a of the case 1 has an elliptical shape. The shape of the detection surface 1a corresponds to an outer peripheral shape of the case 1. The detection surface 1a has an elliptical shape having a first case width W1 along the virtual horizontal axis HL and a second case width W2 along the virtual vertical axis VL. The first case width W1 corresponds to a short diameter of the detection surface 1a. The second case width W2 corresponds to a long diameter of the detection surface 1a. The first case width W1 is different from the second case width W2. When the case 1 has the shape illustrated in FIG. 5, the user M can check the direction of the axis 5A of the motion sensor 5 accommodated in the case 1. The user M can be aware of the direction of the axis 5A of the motion sensor 5 when holding the first measurement apparatus 100a with fingers thereof. When the user M wears the first measurement apparatus 100a at a measurement position that is difficult to visually recognize, such as on the upper arm portion, the user M can easily wear the first measurement apparatus 100a such that the direction of the axis 5A of the motion sensor 5 is in the desired direction. The first case width W1 corresponds to an example of a first width. The second case width W2 corresponds to an example of a second width.

The first case width W1 may be larger or smaller than the second case width W2. The first case width W1 may be smaller than the second case width W2. The user M can easily adjust a direction of the case 1 when the direction of the axis 5A of the motion sensor 5 is set to a predetermined direction.

When the detection surface 1a of the case 1 has an elliptical shape, the side surface 1c of the case 1 is a curved surface. When the first measurement apparatus 100a is attached to the measurement site by a double-sided tape or the like, it is difficult for the first measurement apparatus 100a to be detached due to being caught on clothes and the like. A dimensional ratio of the first case width W1 and the second case width W2, that is, the first case width W1/the second case width W2, may be in a range of 0.2 to 0.8. When the first case width W1/the second case width W2 is less than 0.2, a space for accommodating the optical detection unit 3, the motion sensor 5, and the like is small. When the first case width W1/the second case width W2 is larger than 0.8, the direction of the axis 5A of the motion sensor 5 is hardly recognized when the user M touches the case 1.

The first measurement apparatus 100a includes the optical detection unit 3 including the light-emitting element unit 10 and the light-receiving element unit 20, the motion sensor 5 for detecting the body motion of the user M, and the case 1 having the detection surface 1a facing the user M and accommodating the optical detection unit 3 and the motion sensor 5. In the plan view of the detection surface 1a, the first case width W1 of the case 1 along the virtual parallel axis HL parallel to the axis 5A of the motion sensor 5 is different from the second case width W2 of the case 1 along the virtual vertical axis VL orthogonal to the axis 5A of the motion sensor 5.

The user M can wear the first measurement apparatus 100a such that the axis 5A of the motion sensor 5 is in a predetermined direction.

The first case width W1 may be smaller than the second case width W2.

The user M can easily adjust a direction of the case 1 when the direction of the axis 5A of the motion sensor 5 is set to a predetermined direction.

In the plan view of the detection surface 1a, the detection surface 1a may have an elliptical shape.

The side surface 1c of the case 1 is a curved surface, and the case 1 is hard to catch on clothes and the like of the user M. When the first measurement apparatus 100a is attached to the measurement site by a double-sided tape or the like, the first measurement apparatus 100a is hardly peeled off by the user M.

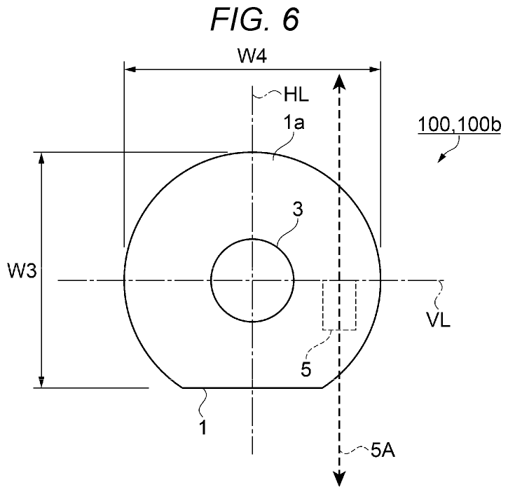

FIG. 6 is a plan view of the detection surface 1a of the measurement apparatus 100. FIG. 6 illustrates a configuration of a second measurement apparatus 100b that is an example of the measurement apparatus 100. FIG. 6 illustrates the detection surface 1a of the case 1 and the optical detection unit 3. In the second measurement apparatus 100b, the motion sensor 5 is accommodated in the case 1. FIG. 6 illustrates the axis 5A, the virtual horizontal axis HL, and the virtual vertical axis VL. In FIG. 6, the light-emitting element unit 10 and the light-receiving element unit 20 in the optical detection unit 3 are omitted.

In a plan view of the detection surface 1a, the detection surface 1a of the case 1 has a shape having a notch in a part of a circle. The notch is provided orthogonal to the virtual parallel axis HL. The shape of the detection surface 1a corresponds to an outer peripheral shape of the case 1. The detection surface 1a has a shape having a third case width W3 along the virtual horizontal axis HL and a fourth case width W4 along the virtual vertical axis VL. The third case width W3 is different from the fourth case width W4. The third case width W3 is smaller than the fourth case width W4. When the case 1 has the shape illustrated in FIG. 6, the user M can check the direction of the axis 5A of the motion sensor 5. The user M can be aware of the direction of the axis 5A of the motion sensor 5 when holding the second measurement apparatus 100b with the fingers thereof. When the user M wears the second measurement apparatus 100b at a measurement position that is difficult to visually recognize, such as on the upper arm portion, the user M can easily wear the second measurement apparatus 100b such that the direction of the axis 5A of the motion sensor 5 is in the desired direction. The third case width W3 corresponds to an example of the first width. The fourth case width W4 corresponds to an example of the second width.

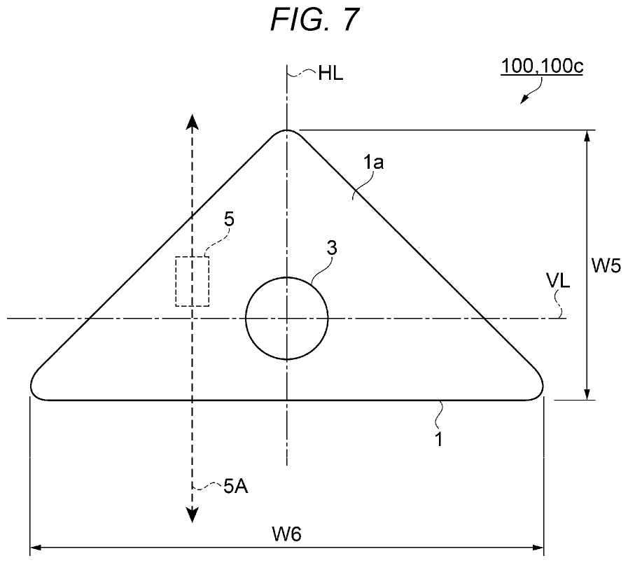

FIG. 7 is a plan view of the detection surface 1a of the measurement apparatus 100. FIG. 7 illustrates a configuration of a third measurement apparatus 100c that is an example of the measurement apparatus 100. FIG. 7 illustrates the detection surface 1a of the case 1 and the optical detection unit 3. In the third measurement apparatus 100c, the motion sensor 5 is accommodated in the case 1. FIG. 7 illustrates the axis 5A, the virtual horizontal axis HL, and the virtual vertical axis VL. In FIG. 7, the light-emitting element unit 10 and the light-receiving element unit 20 in the optical detection unit 3 are omitted.

In the plan view of the detection surface 1a, the detection surface 1a of the case 1 has a triangular shape. A corner portion of the triangular shape has an R shape. The shape of the detection surface 1a corresponds to an outer peripheral shape of the case 1. The detection surface 1a has a shape having a fifth case width W5 along the virtual horizontal axis HL and a sixth case width W6 along the virtual vertical axis VL. The fifth case width W5 is different from the sixth case width W6. The fifth case width W5 is smaller than the sixth case width W6. When the case 1 has the shape illustrated in FIG. 7, the user M can check the direction of the axis 5A of the motion sensor 5. The user M can be aware of the direction of the axis 5A of the motion sensor 5 when holding the third measurement apparatus 100c with the fingers thereof. When the user M wears the third measurement apparatus 100c at a measurement position that is difficult to visually recognize, such as on the upper arm portion, the user M can easily wear the third measurement apparatus 100c such that the direction of the axis 5A of the motion sensor 5 is in the desired direction. The fifth case width W5 corresponds to an example of the first width. The sixth case width W6 corresponds to an example of the second width.

The corner portion may have an R shape. When the corner portion has an R shape, an uncomfortable feeling given to the user M is reduced when the third measurement apparatus 100c is attached to the measurement site.

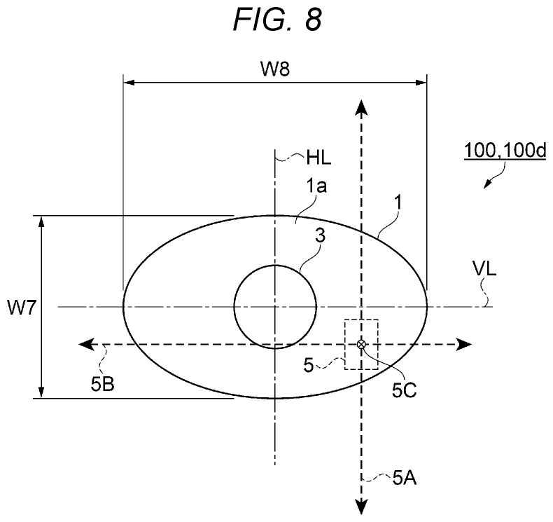

FIG. 8 is a plan view of the detection surface 1a of the measurement apparatus 100. FIG. 8 illustrates a fourth measurement apparatus 100d that is an example of the measurement apparatus 100. FIG. 8 illustrates the detection surface 1a of the case 1 and the optical detection unit 3. In the fourth measurement apparatus 100d, the motion sensor 5 is accommodated in the case 1. FIG. 8 illustrates the axis 5A, a second axis 5B, a third axis 5C, the virtual horizontal axis HL, and the virtual vertical axis VL. In FIG. 8, the light-emitting element unit 10 and the light-receiving element unit 20 in the optical detection unit 3 are omitted.

The fourth measurement apparatus 100d includes an acceleration sensor of three axes including an X axis, a Y axis, and a Z axis as the motion sensor 5. The X axis, the Y axis, and the Z axis represent the axis 5A, the second axis 5B, and the third axis 5C, respectively. The axis 5A and the second axis 5B are on a plane parallel or substantially parallel to the detection surface 1a. The third axis 5C is orthogonal to the detection surface 1a. The second axis 5B is parallel to the virtual vertical axis VL.

In the plan view of the detection surface 1a, the detection surface 1a of the case 1 has an elliptical shape. The shape of the detection surface 1a corresponds to an outer peripheral shape of the case 1. The detection surface 1a has an elliptical shape having a seventh case width W7 along the virtual horizontal axis HL and an eighth case width W8 along the virtual vertical axis VL. The seventh case width W7 corresponds to the short diameter of the detection surface 1a. The eighth case width W8 corresponds to the long diameter of the detection surface 1a. The seventh case width W7 is different from the eighth case width W8. When the case 1 has the shape illustrated in FIG. 8, the user M can check directions of the axis 5A and the second axis 5B of the motion sensor 5. The user M can be aware of the directions of the axis 5A and the second axis 5B of the motion sensor 5 when holding the fourth measurement apparatus 100d with the fingers thereof. When the user M wears the fourth measurement apparatus 100d at a measurement position that is difficult to visually recognize, such as on the upper arm portion, the user M can easily wear the fourth measurement apparatus 100d such that the directions of the axis 5A and the second axis 5B of the motion sensor 5 are in desired directions. The seventh case width W7 corresponds to an example of the first width. The eighth case width W8 corresponds to an example of the second width.

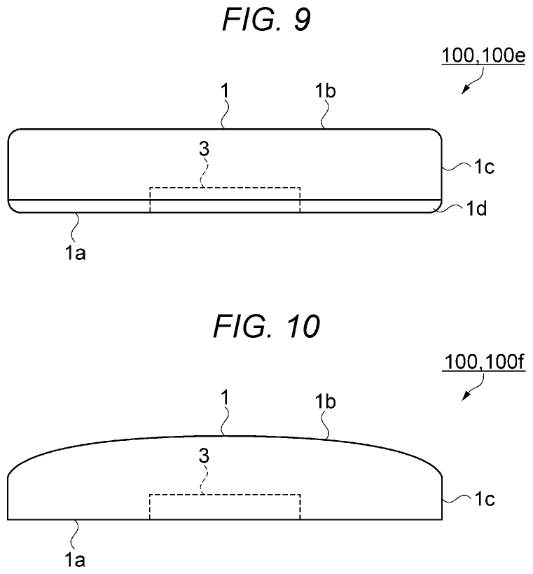

FIG. 9 is a plan view of the side surface 1c of the measurement apparatus 100. FIG. 9 illustrates a schematic configuration of a fifth measurement apparatus 100e that is an example of the measurement apparatus 100. A shape of the detection surface 1a of the fifth measurement apparatus 100e is the same as that of the first measurement apparatus 100a. The shape of the detection surface 1a of the fifth measurement apparatus 100e may be the same as that of the second measurement apparatus 100b or the third measurement apparatus 100c.

The fifth measurement apparatus 100e includes the adhesive layer 1d at at least a part of the detection surface 1a. The adhesive layer 1d is provided at a position of the detection surface 1a in contact with the measurement site of the user M. The adhesive layer 1d is not provided at a position between the optical detection unit 3 and the measurement site of the user M. The adhesive layer 1d allows the user M to wear the fifth measurement apparatus 100e at the measurement site. The adhesive layer 1d includes a base material and an adhesive. Examples of the base material include nonwoven fabric, polyester, polyolefin, and polyurethane. Examples of the adhesive include an acrylic adhesive, a gel adhesive, and a synthetic rubber adhesive. Since the adhesive layer 1d is provided at the detection surface 1a, the fifth measurement apparatus 100e can be attached to any measurement site of the user M. The adhesive layer 1d corresponds to an example of an adhesive layer.

The detection surface 1a may include the adhesive layer 1d.

The user M can wear the fifth measurement apparatus 100e at any measurement site.

FIG. 10 is a plan view of the side surface 1c of the measurement apparatus 100. FIG. 10 illustrates a schematic configuration of a sixth measurement apparatus 100f that is an example of the measurement apparatus 100. A shape of the detection surface 1a of the sixth measurement apparatus 100f is the same as that of the first measurement apparatus 100a. The shape of the detection surface 1a of the sixth measurement apparatus 100f may be the same as that of the second measurement apparatus 100b or the third measurement apparatus 100c.

FIG. 10 illustrates the exterior surface 1b. The exterior surface 1b is a convex curved surface. The sixth measurement apparatus 100f is attached to the measurement site of the user M by using the sleeve 200 which covers the exterior surface 1b, a fixing tape, and the like. The sleeve 200 will be described later. Since the exterior surface 1b is the convex curved surface, a contact area with the sleeve 200 and the likes increases. The sixth measurement apparatus 100f is easily fixed to the measurement site of the user M. In addition, since the exterior surface 1b is a curved surface, the sixth measurement apparatus 100f is hardly caught by the sleeve 200. Due to elasticity of the sleeve 200, an excessive stress is applied to the sixth measurement apparatus 100f, and a movement of the sixth measurement apparatus 100f from the measurement site can be reduced. The exterior surface 1b corresponds to an example of a facing surface.

The exterior surface 1b may be implemented by an elastic member having elasticity. A deviation of the sixth measurement apparatus 100f from the measurement site caused by the stress applied from the sleeve 200 and the likes is reduced. Examples of the elastic member include natural rubber, acrylonitrile rubber, urethane rubber, fluororubber, and silicone rubber.

The case 1 may have the exterior surface 1b facing the detection surface 1a, and the exterior surface 1b may be the convex curved surface.

When the sleeve 200, the fixing tape, and the like cover the exterior surface 1b of the sixth measurement apparatus 100f and is attached to the measurement site of the user M, the deviation of the sixth measurement apparatus 100f from the measurement site of the user M is reduced.

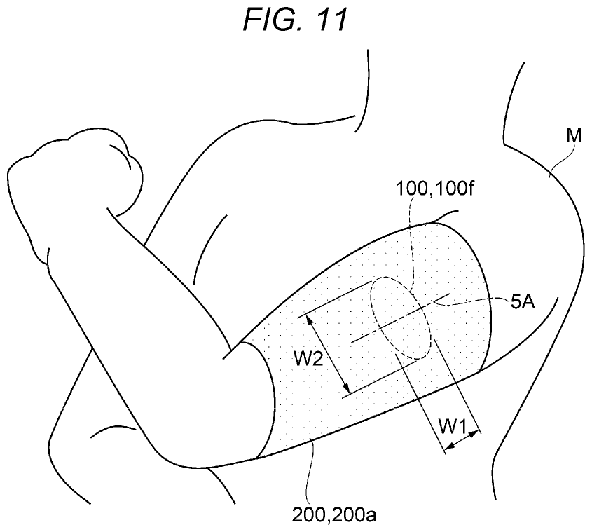

FIG. 11 illustrates the measurement apparatus 100 worn by the user M. FIG. 11 illustrates a state in which the measurement apparatus 100 is attached to the upper arm portion of the user M by the sleeve 200. FIG. 11 illustrates the sixth measurement apparatus 100f and a first sleeve 200a that is an example of the sleeve 200. The first sleeve 200a and the sixth measurement apparatus 100f constitute a wearing set. The wearing set corresponds to an example of a biological information measurement set.

FIG. 11 illustrates the sixth measurement apparatus 100f as the measurement apparatus 100, and the present disclosure is not limited thereto. Any one of the first measurement apparatus 100a, the second measurement apparatus 100b, the third measurement apparatus 100c, and the fourth measurement apparatus 100d may be used instead of the sixth measurement apparatus 100f. The sixth measurement apparatus may be used as the measurement apparatus 100 attached to the sleeve 200. The sleeve 200 corresponds to an example of a wearing tool.

The first sleeve 200a is used when the user M wears the sixth measurement apparatus 100f. The first sleeve 200a covers at least a part of the case 1 of the sixth measurement apparatus 100f. When the user M wears the sixth measurement apparatus 100f at the upper arm portion, a forearm portion, a lower leg portion, a foot portion, or the like, the first sleeve 200a is used. The first sleeve 200a is a cylindrical member. When the first sleeve 200a is attached to the measurement site of the user M, the first sleeve 200a generates a pressing force for pressing the sixth measurement apparatus 100f inward. The first sleeve 200a fixes the sixth measurement apparatus 100f to the measurement site by the pressing force.

The first sleeve 200a covers the case 1 of the sixth measurement apparatus 100f and presses the sixth measurement apparatus 100f to the measurement site of the user M. By using the sixth measurement apparatus 100f in which the first case width W1 and the second case width W2 are different from each other, the user M can align the direction of the axis 5A of the motion sensor 5 with a predetermined direction. For example, the user M aligns the virtual horizontal axis HL corresponding to the first case width W1 in a longitudinal direction of the upper arm portion. The user M can match or substantially match the axis 5A of the motion sensor 5 with the longitudinal direction of the upper arm portion. The user M aligns the virtual vertical axis VL corresponding to the second case width W2 in a circumferential direction of the upper arm portion. The user M can match or substantially match the axis 5A of the motion sensor 5 with the longitudinal direction of the upper arm portion. The user M can match or substantially match the axis 5A of the motion sensor 5 with the longitudinal direction of the upper arm portion in a state in which the sixth measurement apparatus 100f cannot be directly recognized visually.

The wearing set includes the sixth measurement apparatus 100f and the first sleeve 200a. The sixth measurement apparatus 100f includes the optical detection unit 3 including the light-emitting element unit 10 and the light-receiving element unit 20, the motion sensor 5 for detecting the body motion of the user M, and the case 1 having the detection surface 1a facing the user M and accommodating the optical detection unit 3 and the motion sensor 5. The first sleeve 200a covers the case 1 and presses the sixth measurement apparatus 100f to the user M. In the plan view of the detection surface 1a, the first case width W1 of the case 1 along the virtual parallel axis HL parallel to the axis 5A of the motion sensor 5 is different from the second case width W2 of the case 1 along the virtual vertical axis VL orthogonal to the axis 5A of the motion sensor 5.

The user M can align the direction of the axis 5A of the motion sensor 5 with a predetermined direction. The user M can match or substantially match the axis 5A of the motion sensor 5 with the longitudinal direction of the upper arm portion in a state in which the sixth measurement apparatus 100f cannot be directly recognized visually.

The exterior surface 1b in contact with the first sleeve 200a of the case 1 may be an elastic member.

A deviation of the sixth measurement apparatus 100f from the measurement site caused by a stress applied from the first sleeve 200a is reduced.

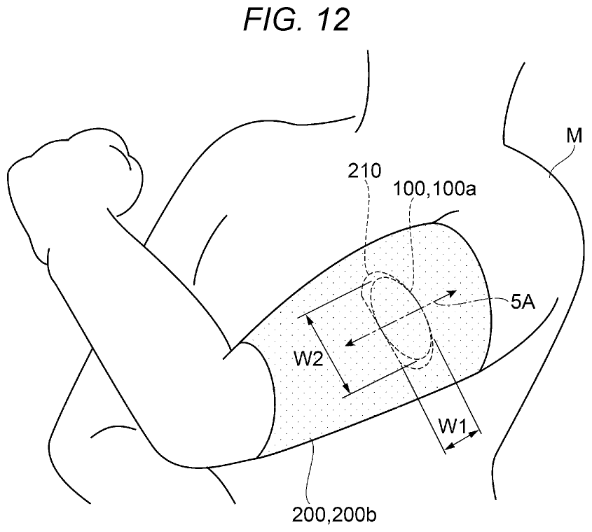

FIG. 12 illustrates the measurement apparatus 100 worn by the user M. FIG. 12 illustrates a state in which the measurement apparatus 100 is attached to the upper arm portion of the user M by the sleeve 200. FIG. 12 illustrates the first measurement apparatus 100a and a second sleeve 200b that is an example of the sleeve 200. In FIG. 12, the second sleeve 200b and the first measurement apparatus 100a constitute a wearing set.

FIG. 12 illustrates the first measurement apparatus 100a as the measurement apparatus 100, and the present disclosure is not limited thereto. Any one of the second measurement apparatus 100b, the third measurement apparatus 100c, the fourth measurement apparatus 100d, the fifth measurement apparatus 100e, and the sixth measurement apparatus 100f may be used instead of the first measurement apparatus 100a.

The second sleeve 200b is used when the user M wears the first measurement apparatus 100a. The second sleeve 200b includes a pocket 210 into which the first measurement apparatus 100a can be inserted. The pocket 210 holds the first measurement apparatus 100a. A shape of the pocket 210 coincides with or substantially coincides with the shape of the first measurement apparatus 100a. When the first measurement apparatus 100a is inserted into the pocket 210, the direction of the axis 5A of the motion sensor 5 is set to the desired direction. When the user M inserts the first measurement apparatus 100a into the pocket 210, the first measurement apparatus 100a is held by the second sleeve 200b in a posture in which the axis 5A of the motion sensor 5 coincides with or substantially coincides with the longitudinal direction of the upper arm portion. The second sleeve 200b has the same configuration as the first sleeve 200a except that the second sleeve 200b includes the pocket 210.

What is claimed is:

1. A biological information measurement apparatus comprising:
a detection unit including a light-emitting unit and a light-receiving unit;
an inertial sensor configured to detect a body motion of a test subject; and
a housing having a measurement surface configured to face the test subject and accommodating the detection unit and the inertial sensor, wherein
in a plan view of the measurement surface, a first width of the housing along a parallel axis parallel to an axis of the inertial sensor is different from a second width of the housing along an orthogonal axis orthogonal to the axis of the inertial sensor.

2. The biological information measurement apparatus according to claim 1, wherein
the first width is smaller than the second width.

3. The biological information measurement apparatus according to claim 1, wherein
the measurement surface includes an adhesive layer.

4. The biological information measurement apparatus according to claim 1, wherein
the housing has a facing surface facing the measurement surface, and
the facing surface is a convex curved surface.

5. The biological information measurement apparatus according to claim 1, wherein
the measurement surface has an elliptical shape in the plan view of the measurement surface.

6. A biological information measurement set comprising:
a biological information measurement apparatus including
a detection unit including a light-emitting unit and a light-receiving unit,
an inertial sensor configured to detect a body motion of a test subject, and
a housing having a measurement surface configured to face the test subject and accommodating the detection unit and the inertial sensor; and
a wearing tool configured to cover the housing and press the biological information measurement apparatus to the test subject, wherein
in a plan view of the measurement surface, a first width of the housing along a parallel axis parallel to an axis of the inertial sensor is different from a second width of the housing along an orthogonal axis orthogonal to the axis of the inertial sensor.

7. The biological information measurement set according to claim 6, wherein
a facing surface of the housing in contact with the wearing tool is implemented by an elastic member.
